(19) United States
(12) Patent Application Publication
(10) Pub. No.: US 2007/0259959 A1
(43) Pub. Date: Nov. 8, 2007

(54) Ppar Agonists for the Treatment of Hcv Infection

(76) Inventors: Riccardo Cortese, Rome (IT); Alfredo Nicosia, Rome (IT); Alessandra Vitelli, Rome (IT)

Correspondence Address: MERCK AND CO., INC, P O BOX 2000, RAHWAY, NJ, 07065-0907, US

(21) Appl. No.: 10/579,509

(22) PCT Filed: Nov. 17, 2004
(86) PCT No.: PCT/EP04/13067

§ 371 Date: Apr. 9, 2007

(30) Foreign Application Priority Data
Nov. 20, 2003 (GB) 0327050.1

(51) Int. Cl. A61K 31/192; A61P 31/12

(52) U.S. Cl. 514/570

(57) ABSTRACT

The present invention concerns the use of PPARα agonists in methods and compositions for the treatment or prevention of infection by hepatitis C virus (HCV) in mammals, especially humans.

The present invention concerns methods and compositions useful in the therapeutic treatment of mammals, especially humans. In particular, the invention concerns methods and compositions for treatment or prevention of infection by the hepatitis C virus (HCV).

HCV is a major human pathogen, infecting about 3 percent of the world's population, and is a major cause of liver disease. A striking feature of HCV infection is the tendency towards a chronic status leading to liver diseases such as chronic hepatitis, cirrhosis and hepatocellular carcinoma. HCV infection is also implicated in mixed cryoglobulinemia, a B-lymphocyte proliferative disorder.

A major obstacle in understanding the mechanism of HCV infection and in the design and testing of appropriate therapies is the lack of knowledge of the HCV cellular receptors and the mechanisms by which they mediate viral attachment and entry to cells. At least three different receptors have been implicated, namely the low density lipoprotein (LDL) receptor (Agnello et al, PNAS, 1999, 96, 12766-71); the CD81 receptor (Pileri et al, Science, 1998, 282, 938-41); and the scavenger receptor type B class I (SRB1) (WO 03/040726, Scarselli et al, EMBO, 2002, 12, 58017-25 and Bartosch et al, J. Biol. Chem., 2003, 278, 41624-30).

Peroxisome proliferator receptors (PPARs) form part of the nuclear receptor superfamily and are in involved in the control of lipid metabolism. They exist as α, β, γ, and δ subtypes (for a review, see Desvergne and Wahli, Endocrine Reviews, 1999, 20, 649-88). PPAR activation has been linked to diverse phenomena such as fatty acid metabolism, inflammatory responses, atherosclerosis and control of the cell cycle. However, there has hitherto been no disclosure of a link between PPAR activity and HCV infection.

According to the present invention, there is provided the use of a PPARα agonist for the manufacture of a medicament for treatment or prevention of HCV infection in a mammal.

There is further provided a method of treating or preventing HCV infection in a mammalian subject comprising administration to that subject of a therapeutically effective amount of a PPARα agonist. Typically, the mammalian subject is human.

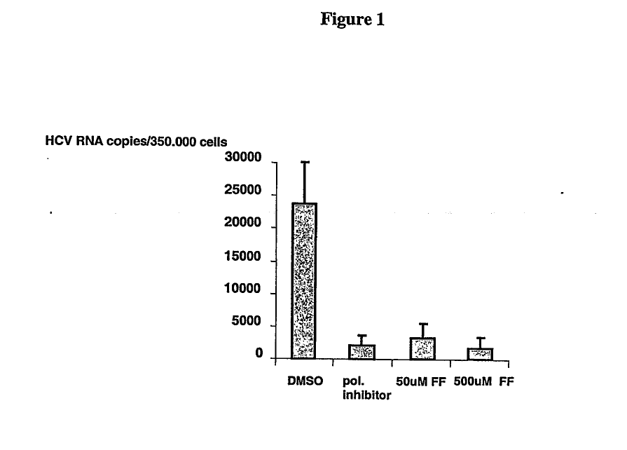

FIG. 1 shows the numbers of copies of HCV RNA detected when cultured human hepatocytes were incubated with serum from an HCV-infected patient in the presence and absence of fenofibric acid.

It is believed that PPARα agonism has the effect of inhibiting entry of HCV to hepatocytes, possibly through a reduction of the expression and/or cell surface display of SRB1. Such effect is useful both in preventing infection by HCV in the first place and in arresting the progress of an existing infection by preventing further cells from becoming infected. Thus, in accordance with the invention, the PPARα agonist may usefully be administered to subjects at risk of contracting HCV infection (prophylaxis) or to subjects who have already contracted HCV infection (active treatment).

According to a further aspect of the invention, there is provided a method of inhibiting entry of HCV to a cell comprising contacting said cell with a PPARα agonist. Preferably the cell is a hepatocyte.

In principle, any compound known or discovered to have PPARα agonist activity may be used in the invention, but compounds suitable for oral administration are preferred. Compounds having PPARα agonist activity may be identified using published assay methods such as the cell-based transactivation assay described in Berger et al, J. Biol. Chem., 1999, 274, 6718-25. Suitable compounds include those which are selective PPARα agonists and those which combine activity at the alpha receptor with activity at one or more of the other subtypes, e.g. PPARα/γ dual agonists. Known selective PPARα agonists include fenofibrate, beclofibrate, bezafibrate, ciprofibrate, clofibrate, etofibrate, other fibric acid derivatives, gemcarbene, gemfibrozil, GW 7647, BM 170744, LY 518674, Atromid™, Lopid™ and Tricor™, as well as compounds disclosed in Adams et al Bioorg. Med Chem. Lett., 2003, 13, 3185-90. Examples of PPARα/γ dual agonists include include KRP-297 (K-0767), muraglitazar (BMS-298585), farglitazar, ragaglitazar, tesaglitazar (AZ-242), JT-501, GW-2570, GI-262579, CLX-0940, GW-1536, GW-1929, GW-2433, L-796449, LR-90, SB-219994, LY-578, LY-4655608, LSN-862, LY-510929 and LY-929, as well as compounds disclosed in Desai et al Bioorg. Med Chem. Lett., 2003, 13, 35414 and in Desai et al Bioorg. Med Chem. Lett., 2003, 13, 2795. Further disclosure of selective PPARα agonists or PPARα/γ dual agonists appears in WO 97/28115, WO 00/78312, WO 00/78313, WO 00/196321, WO 00/181327, WO 00/134148, WO 02/064094, WO 02/060434, WO 02/26729, WO 01/60807, EP1194147, EP1194146, WO 03/066581 and WO 03/075911.

Preferred compounds for use in the invention include fenofibrate, bezafibrate, ciprofibrate, gemfibrozil and MK-0767.

The PPARα agonist may be administered alone or in combination with one or more additional therapeutic agents known to be useful in the treatment or prevention of HCV infection or the symptoms thereof. Examples of such additional therapeutic agents include interferon-a pegylated interferon-α, ribavirin, HCV NS3 protease inhibitors, HCV polymerase inhibitors, anti-HCV antibodies and HCV vaccines. As used herein, the expression “in combination with” requires that therapeutically effective amounts of both the PPARα agonist and the additional therapeutic agent are administered to the subject, but places no restriction on the manner in which this is achieved. Thus, the two species may be combined in a single dosage form for simultaneous administration to the subject, or may be provided in separate dosage forms for simultaneous or sequential administration to the subject. Sequential administration may be close in time or remote in time, e.g. one species administered in the morning and the other in the evening. The separate species may be administered at the same frequency or at different frequencies, e.g. one species once a day and the other two or more times a day. The separate species may be administered by the same route or by different routes, e.g. one species orally and the other parenterally, although oral administration of both species is preferred, where possible. When the additional therapeutic agent is a vaccine or antibody, it will typically be administered parenterally and separately from the PPARα agonist.

In a further aspect, the invention provides a pharmaceutical composition or kit comprising, in the same or separate pharmaceutically acceptable carriers, a PPARα agonist and one or more therapeutic agents selected from interferon-α, pegylated interferon-α, ribavirin, HCV NS3 protease inhibitors, HCV polymerase inhibitors, anti-HCV antibodies and HCV vaccines. Kits comprising separately-formulated therapeutic agents will typically comprise instructions for the separate administration of the therapeutic agents.

The PPARα agonists and optional additional therapeutic agent(s) are typically used in the form of pharmaceutical compositions comprising the relevant active ingredient(s) and a pharmaceutically acceptable carrier. Where the active ingredient comprises an acidic or basic group, said ingredient may be in the form of the free acid or base or in the form a pharmaceutically acceptable salt. Preferably the pharmaceutical compositions are in unit dosage forms such as tablets, pills, capsules, powders, granules, sterile parenteral solutions or suspensions, metered aerosol or liquid sprays, drops, ampoules, transdermal patches, auto-injector devices or suppositories; for oral, parenteral, intranasal, sublingual or rectal administration, or for administration by inhalation or insufflation. The principal active ingredient typically is mixed with a pharmaceutical carrier, e.g. conventional tableting ingredients such as corn starch, lactose, sucrose, sorbitol, talc, stearic acid, magnesium stearate and dicalcium phosphate, or gums, dispersing agents, suspending agents or surfactants such as sorbitan monooleate and polyethylene glycol, and other pharmaceutical diluents, e.g. sterile water, to form a homogeneous preformulation composition containing a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This preformulation composition is then subdivided into unit dosage forms of the type described above containing from 0.1 to about 500 mg of the active ingredient of the present invention. Typical unit dosage forms contain from 1 to 100 mg, for example 1, 2, 5, 10, 25, 50 or 100 mg, of the active ingredient. Tablets or pills of the composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compositions useful in the present invention may be incorporated for administration orally or by injection include aqueous solutions, liquid- or gel-filled capsules, suitably flavoured syrups, aqueous or oil suspensions, and flavoured emulsions with edible oils such as cottonseed oil, sesame oil or coconut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, poly(ethylene glycol), poly(vinylpyrrolidone) or gelatin.

For treating or preventing HCV infection, a suitable dosage levels of the PPARα agonist are similar with published values for the compounds concerned when used for other therapeutic purposes (e.g. control of lipid levels), or may be determined by methods known to those skilled in the art. Typical levels are in the range of about 0.01 to 250 mg/kg per day, preferably about 0.01 to 100 mg/kg per day, and more preferably about 0.05 to 50 mg/kg of body weight per day, of the active compound. Any suitable dosing regimen may be used, e.g. 1-4 times daily.

A suitable dose of fenofibrate is 100-200 mg per adult person daily.

### EXAMPLES

### Example 1

### Inhibition of HCV Infection of Cultured Human Hepatocytes by Fenofibric Acid

Isolated human hepatocytes from surgical liver resection were seeded in 24 well microplates at the density of 3×10⁵ cells/well. Cells were allowed to attach and recover 24 hours and then medium was replaced with a fresh one containing different concentrations of fenofibric acid (50 μM and 500 μM). Hepatocytes were incubated 24 hours with the indicated amounts of fenofibric acid, then medium was replaced with fresh one containing the same amounts of fenofibric acid and a fixed amount (100 μl) of an infectious human serum from a patient chronically infected with HCV. Cells were incubated 18 hours with the virus to allow infection, then washed and incubated for four days. Total RNA was extracted and viral replication was measured by quantitative RT-PCR.

Typically, 10⁴ to 10⁵ copies of genomes per well are detected after four days from infection. To be sure that the measured viral RNA derived from active replication, a small molecule inhibitor of the viral replicase was included as a positive control.

Viral replication was measured on total RNA by quantitative PCR and expressed as number of HCV copies/350,000 cells. The experiment was performed in triplicate wells and values are shown with standard deviations. Fenofibric acid was dissolved in DMSO and tested at 50 μM and 500 μM. Final concentration of DMSO in the assay was 0.5%, therefore all the control infections (not-inhibited and with the HCV replicase inhibitor) were done in the presence of 0.5% DMSO. The results are shown in FIG. 1, and it is clear that fenofibric acid reduced the infectivity by up to 90%.

### Example 2

A hard gelatine capsule containing 100 mg of fenofibrate may be administered orally to a 60 Kg adult patient in need thereof for the treatment of HCV infection. Such administration may take place twice or three times a day.

1. (canceled)

2. A method of treating or preventing HCV infection in a mammalian subject comprising administration to that subject of a therapeutically effective amount of a PPARα agonist.

3. The method according to claim 2 wherein the PPARα agonist is administered in combination with one or more therapeutic agents selected from interferon-α, pegylated interferon-α, ribavirin, a HCV NS3 protease inhibitor, a HCV polymerase inhibitor, anti-HCV antibodies and a HCV vaccine.

4. The use according to claim 1 or the method according to claim 2 wherein the mammal is a human.

5. A method of inhibiting entry of HCV to a cell comprising contacting said cell with a PPARα agonist.

6. The method according to claim 5 wherein the cell is a hepatocyte.

7. A pharmaceutical composition comprising a PPARα agonist and a pharmaceutically acceptable carrier in combination with one or more therapeutic agents selected from interferon-α, pegylated interferon-α, ribavirin, a HCV NS3 protease inhibitor, a HCV polymerase inhibitor, anti-HCV antibodies and a HCV vaccine.

8. A kit comprising a PPARα agonist and one or more therapeutic agents selected from interferon-α, pegylated interferon-α, ribavirin, a HCV NS3 protease inhibitor, a HCV polymerase inhibitor, anti-HCV antibodies and a HCV vaccine, for simultaneous or sequential administration.

9. The method according to claim 2 wherein the PPARα agonist is a selective PPARα agonist.

10. The method according to claim 2 wherein the PPARα agonist is a PPARα/γ dual agonist.

11. The method according to claim 2 wherein the PPARα agonist is fenofibrate, bezafibrate, ciprofibrate, gemfibrozil or MK-0767.

12. The method according to claim 3, wherein the mammal is a human.

13. The method according to claim 5 wherein the PPARα agonist is a selective PPARα agonist.

14. The method according to claim 5 wherein the PPARα agonist is PPARα/γ dual agonist.

15. The method according to claim 5 wherein the PPARα agonist is fenofibrate, bezafibrate, ciprofibrate, gemfibrozil or MK-0767.

16. The pharmaceutical composition according to claim 7 wherein the PPARα agonist is a selective PPARα agonist.

17. The pharmaceutical composition according to claim 7 wherein the PPARα agonist is PPARα/γ dual agonist.

18. The pharmaceutical composition according to claim 7 wherein the PPARα agonist is fenofibrate, bezafibrate, ciprofibrate, gemfibrozil or MK-0767.

19. The kit according to claim 8 wherein the PPARα agonist is a selective PPARα agonist.

20. The kit according to claim 8 wherein the PPARα agonist is PPARα/γ dual agonist.

21. The kit according to claim 8 wherein the PPARα agonist is fenofibrate, bezafibrate, ciprofibrate, gemfibrozil or MK-0767.
